(19) United States
(12) Patent Application Publication
(10) Pub. No.: US 2010/0144919 A1
(43) Pub. Date: Jun. 10, 2010

(54) Reactive polymeric nanoparticles (RPNPS) for restoration materials in dentistry

(76) Inventors: Janos Borbely, Debrecen (HU); Melinda Szaloki, Nyiradony (HU); Andrea Uveges, Debrecen (HU); Katalin Bukovinszki, Debrecen (HU); Csaba Hegedus, Debrecen (HU)

Correspondence Address: Thomas A. O'Rourke;Bodner & O'Rourke, LLP, Suite 108, 425 Broadhollow Road, Melvillle, NY, 11747, US

(21) Appl. No.: 11/998,154

(22) Filed: Nov. 28, 2007

(51) Int. Cl. A61K 6/083; C08F 220/06; C08F 4/30; C08F 4/04

(52) U.S. Cl. 523/116; 526/318.6; 526/317.1; 526/229; 526/219.6

(57) ABSTRACT

A method is disclosed in which modified and generic dental resins are combined as mixtures with reactive polymeric nanoparticles (RPNPs). Nanoparticles as additives clearly demonstrate that the RPNPs significantly influence the mechanical and shrinkage properties of the matrix and composites.

The present invention claims priority on U.S. Provisional Patent Application Ser. No. 60/861,208 filed Nov. 27, 2006, the disclosures of which are incorporated herein by reference.

### FIELD OF THE INVENTION

The present invention is directed to dental filling materials made from nanocomposites, i.e., reactive polymeric nanoparticles (RPNP's). More particularly, the present invention is directed to an improved modified dental resin that has been reacted with a nanoparticle containing reactive polymeric nanoparticles. The present invention relates to reactive nanoparticles prepared by, for example, a free radical non-linear copolymerization of mono/di/tri and multivinyl acrylic monomers in a miniemulsion and/or solution.

### BACKGROUND OF THE INVENTION

There are two general types of dental restorations. One type is called direct restoration which is done by inserting filling material directly into the tooth. The other type of dental restoration is indirect restoration where the fabrication is done outside the mouth. Traditionally, the direct restorations have been done with gold, base metal alloys and dental amalgam. Dental amalgam is a mixture of mercury and silver alloy that forms a hard solid metal filling. It is self hardening at mouth temperatures. In recent years there has been an increase in the development of more aesthetic materials made of ceramic and plastic. These can be designed to more closely match the color of natural teeth but many of these more aesthetic materials are not as strong as the more traditional materials.

Other materials that are being used in direct dental restorations are composites which can be a mixture of submicron glass filler and acrylic that forms a solid tooth colored restoration. These are self or light hardening at the patient's mouth temperature. Glass ionomers have also been used in dental restorations. These are typically a self hardening mixture of fluoride containing glass powder and an organic acid that forms a solid tooth colored restoration that is able to release fluoride. Another type of restorative material is a self or light hardening mixture of submicron glass filler with fluoride containing glass powder and acrylic resin that forms a solid tooth colored restoration able to release fluoride.

Amalgams containing mercury have been under fire recently as people have been concerned about the toxicity of mercury. The other materials used in direct dental restoration have other issues as well. The composites of glass fillers and acrylic are good in small to medium size restorations, but less effective in larger restorations. The fluoride and glass powder mixtures with organic acids are used primarily for small non load bearing fillings, cavity liners and cements for crowns and bridges. These materials are not usually suitable for load bearing restorations. Resin ionomer restorations that include a blend of submicron glass filler fluoride containing glass powder and acrylic resins are also only suitable in non load bearing restorations where indirect restorations are usually comprised of other materials. These can include porcelain, ceramic or glass like fillings and crowns. These materials can be brittle and can fracture under heavy biting loads. Another indirect restoration material is porcelain fused to metal. These restorations are stronger than the ones without metal. However, both porcelain types can wear down opposite teeth if the porcelain becomes rough. The porcelain portion can still be subject to fractures. Gold alloys and base metal alloys are usually fairly strong and do not suffer from corrosion or fracture. These materials, however, transmit heat and cold and can be uncomfortable in certain situations. In addition, these materials do not resemble natural teeth in their coloration.

Acrylic resins can be in a variety of forms, including those used in dental restorations. One form is an acrylic matrix (matrix phase). Another is as filler particles (dispersed phase) and a coupling phase which coats the filler particles allowing them to couple with the matrix phase. One resin that is commonly used in dental restorations is known as bis-GMA, which is produced by a reaction between bisphenol A and a glycidal methacrylate. The resin is a dimethacrylate monomer which is induced to polymerize by the presence of free radicals. These free radicals can be generated by a chemical reaction or the introduction of heat or light.

Light activated resins come as a paste in a syringe or compule. The paste contains a photoinitiator molecule such as camphoroquinone and an amine activator. When exposed to light in the 400 to 500 nm wavelength range, the photoinitiator becomes excited and reacts with the amine to produce the free radicals, thereby initiating the polymerization process.

One of the major problems with composite restorative materials is polymerization shrinkage. As the resin matrix polymerizes, the organized polymer molecules occupies less space than its disorganized constituent monomers did. As the composite cures, it shrinks and may pull away from the cavity walls. Volumetric shrinkage can compromise marginal seals and rupture adhesive bonds created at the tooth restorative interface. Additionally, allergic reactions generated by residual monomers may affect some patients.

In the area of dental filling materials, polymeric nanoparticles have been the subject of great interest. For dental filling materials, multivinyl monomers were recently crosslinked in situ by a photopolymerization process. Dental resin composites comprise a blend of hard, inorganic particles bound together by a soft, resin matrix. During the photopolymerization, the conversion of the monomer molecules of the matrix into a polymer network is accompanied by a closer packing of the molecules, which leads to bulk contraction. (Refs 6-8). Polymerization shrinkage of dental resin composites is due to the fact that monomers are converted into a polymer network and therefore exchange(s) van der Waals spaces in covalent bond spaces. This polymerization shrinkage creates contraction stress in the resin composite and internal stress and deformation in the surrounding tooth structure (Ref 9).

Polymerization shrinkage is a critical limitation of dental composite resins. Stresses from shrinkage can cause clinical problems such as postoperative pain, fracture of the tooth, and opening of restoration margins that can result in microleakage and recurrent carries (Refs 10-15). Many factors have a direct effect on the polymerization shrinkage of composite resin: curing method (chemical or light-curing), placement technique (incremental or bulk), cavity configuration and size of the restoration.

Since the introduction of dental resin composites, reduction of the polymerization shrinkage has been an important issue. Non-shrinking resins and modified resin filler particles have been developed to tackle this problem, but are not yet commercially available. Polymerization shrinkage, contraction stress, elastic modulus, and flow are important factors determining, the final properties of the resin composite. However, so far, only, polymerization shrinkage in relation to elastic modulus and polymerization contraction stress in relation to elastic modulus and filler load and type of resin composite have been evaluated.

In commercially available composite materials the ratio of inorganic filler is commonly about 50-80% and they are commonly used for dental restorations. Current composite materials contain crosslinking acrylates or methacrylates, inorganic fillers such as glass or quartz, and a photoinitiator system suitable for curing by visible light. Typical methacrylate monomers of the dental resin include 2,2′-bis[4-(2-hydroxy-3-methacryloyloxypropyl)phenyl]propane (Bis-GMA); ethoxylated Bisphenol A dimethacrylate (EBPDMA); 1,6-bis-[2-methacryloyloxyethoxycarbonylamino]-2,4,4-trimethylhexane (UDMA); dodecanediol dimethacrylate and triethylene glycol dimethacrylate (TEGDMA).

One of the more common commercially used monomers is Bis-GMA, which is an especially important monomer in dental resins. However, it is highly viscous at room temperature and is insufficiently converted to polymer when it is cured. To reach the optimal consistency of dental material it is, therefore, diluted with a second, lower viscosity polymerizable component, typically an acrylate or methacrylate monomer, such as trimethylol propan trimethacrylate (TMPTMA), 1,6-hexanediol dimethacrylate (HDDMA), 1,3-butanediol dimethacrylate (BDDMA), ethylene glycol dimethacrylate (EGDMA), diethylene glycol dimethacrylate (DEGDMA), triethylene glycol dimethacrylate (TEGDMA), or tetraethylene glycol dimethacrylate. However, while providing low viscosity, lower viscosity components (generally low molecular weight monomers) contribute to increased shrinkage. Increasingly, Bis-GMA and TEGDMA are combined with UDMA and EBPDMA, but shrinkage remains high enough that improvement is desirable.

An advantage of dental composite materials is their superior cosmetic appearance compared to the traditional metal amalgam. Dental filling amalgams have longevity because, of the improvement of the dental material. The primary reasons for failure, when it occurs, are excessive shrinkage during photopolymerization in the tooth cavity, which causes leakage and bacterial reentry.

Another disadvantage of present-day dental composites is their inadequate strength and toughness, as reflected in the measured properties of flexural strength and fracture toughness. Hence, there is still a need for new monomers and new monomer combinations which, when polymerized, impart high fracture toughness and flexural strength in the resulting composite. It is also highly desirable to have low shrinkage and low shrinkage stress on polymerization.

The properties of the filler material that exert the greatest influence are particle size and distribution of inorganic particles and concentration of inorganic filler in the dental composite. When the resin component is high, the composites have low shrinkage values, high contraction stress values and are relatively rigid. Low-viscosity or flowable resin composites have high shrinkage values, low contraction stress values and are relatively flexible.

The focus of this invention is the modification of acrylate-based dental resins to reduce the shrinkage at optimal viscosity. The final mechanical properties are dependent upon the inorganic filler (Ref 16). In this invention the dental resin is modified by the reactive polymer-based nanoparticles. The nanoparticles are formed by the reaction product of styrenic and dental acrylic (modified dental resin as model composition because of the styrene) and/or only dental acrylic monomers (generic dental resin). The properties of the nanoparticle can be influenced at the molecular level, e.g. cross-linking. The nanoparticle contains reactive pendant double bounds and these are subject to further polymerization (Refs 17, 18). It was expected that the modified resin will show lower shrinkage and its mechanical properties will be improved, than the commercial dental materials. It is required that the nanoparticles and the resin be compatible, i.e. the nanoparticles have to solve or swell well in the mixture of monomers, and that the pendant double bounds of RPNPs must react with the monomers by photopolymerization.

Ruppert, U.S. Pat. No. 4,085,34, claims composite material having a low shrinkage force. The object of this invention is to supply a composite material for dental applications which reduces the risk of detachment of the restorative from the cavity wall. According to this invention, the use of non-agglomerated nanofillers, a filler mixture, various monomer mixtures and a reduction of the content of the photoinitiator, results in lower shrinkage.

Jin, U.S. Pat. No. 6,968,72, disclosed low shrinkage, polymerizable oligomers comprising units of the structure which consist of A: an organic radical comprising 1 to about 6 (meth)acrylate groups and 0 to about 5 hydroxy groups and B: an organic radical comprising 1 to about 5 epoxide groups wherein A and B are linked through the reaction of an epoxide and hydroxyl group. According to the claims, the polymerizable dental resin (A+B) composition has volume shrinkage of less than about 2%.

Bowman, U.S. Pat. No. 5,766,35, the shrinkage of photopolymerizable polymer based dimethacrylate system is compared to thiol-ene system. The traditional dimethacrylate-based resins have significantly higher volumetric shrinkage during the polymerization process which can cause tooth-composite adhesive failure, microleakage and recurrent caries. This reduces the longevity and utility of current dental restorative composites. In the case of the thiol-ene system, the final double bond conversion is increased and reduces the reactive monomer in the cured dental material resulting in reduced shrinkage.

Arthur, U.S. Pat. No. 225,228, relates to composite materials for restorative dentistry, more particularity new components, which acquaint an attractive combination of good mechanical properties and low shrinkage. Synthesis of more unsaturated (meth)acrylate monomers are presented in the examples part of this invention. Prepared hyperbranched polyester metacrylate by capping a hyperbranched polyester polyol with methacrylic anhydride is disclosed to form new dental composite, thus to combine reduced shrinkage with sufficiently low viscosity high polymerization rate and acceptable mechanical properties.

### OBJECTS OF THE INVENTION

It is an object of the invention to provide an improved modified and generic dental resin with reactive polymeric nanoparticles.

It is also an object of the invention to provide an improved nanoparticle that is cross-linked.

It is another object of the invention to provide a polymeric nanoparticle made from a free radical non-linear copolymerization process.

It is a still further object of the invention to provide a reactive polymeric nanoparticle that is made from a mono/di/tri and multivinyl acrylic monomer.

It is still another object of the invention to provide reactive polymeric nanoparticles made in a miniemulsion and/or solution.

It is a still further object of the invention to provide a reactive polymeric nanoparticle that is suitable for a variety of applications.

It is also an object of the invention to provide a reactive polymeric nanoparticle that is suitable for use as additives in dental resin.

It is another object of the invention to provide a modified dental resin that decreases shrinkage of dental materials.

It is still another object of the invention to provide a method for making modified dental resin and material.

### SUMMARY OF THE INVENTION

A nanocomposite is made by reacting a monomer comprised of a mono/di/tri and multi-vinyl monomer with a reactive polymeric nanoparticle. The monomer is preferably an acrylic monomer. The reactive polymeric nanoparticles are formed from, for example, a monomer having at least one preferably two double bonds. The monomer is preferably a vinyl monomer. The other material is preferably an acrylic or styrenic based compound having two or more double bonds. In a preferred embodiment, styrene and ethyleneglycol-dimethacrylate reacted together by free radical non-linear copolymerization. Once the reactive polymeric nanoparticle is formed, it is reacted with a resin composed vinyl monomers. Preferably the resin is a commercial dental resin. The modified resin containing the reactive polymeric nanoparticles is polymerized preferably by photopolymerization. More preferably, the photopolymerization occurs in blue light.

The RPNP's can be formed in, for example, emulsion and/or solution by a free radical non-linear copolymerization of mono/di/tri and multi-vinyl acrylic monomers. The nanoparticles so formed may be cross-linked and possess double bonds. Preferably acrylic or styrenic monomers are used. The RPNP containing nanocomposites are blended with a dental resin.

The present invention is directed broadly to modified and generic dental resins prepared, for example, by a mixing monomers such as a mono/di/tri and multivinyl monomer with a reactive polymeric nanoparticles (RPNPs). The monomer is preferably an acrylic monomer.

RPNPs are formed in emulsion, such as a miniemulsion (Refs 1-3) and/or in solution (Refs 4, 5) by a free radical non-linear copolymerization of mono/di/tri and multivinyl acrylic monomers. The nanoparticles so formed may be crosslinked and possess pendant double bonds.

Acrylic or styrenic monomers are used. The applied monomers, styrenic and acrylic monomers are added to the modified dental resin and to the generic resin. Acrylic dental monomers were used to prepare RPNPs. (Ref 19-22).

The nanocomposites (NCs) were made by mixing monomers with the reactive polymeric nanoparticles (RPNPs). The nanoparticles can swell in the mixtures of monomers which are normally applied as cross-linkers.

Organofiller of modified resin was synthesized by forming styrene-ethylene glycol dimetacrylate (ST-EGDMA; Type-1) in solution and styrene-trimethylol propane trimethacrylate (ST-TMPTMA; Type-2) in miniemulsion reactive polymeric nanoparticles by free radical non-linear copolymerization. The prepared nanoparticles have different swellable features due to the method of the preparation.

Borbely, U.S. application Ser. No. 11,482,540 the synthesis of styrene-trimethylol propane trimetacrylate (ST-TMPTMA) copolymer in miniemulsion was introduced. Due to the ester likage of the TMPTMA it is a water-in-oil emulsion. Accordingly, the size of nanoparticles before precipitation is increased by the increasing of amount of crosslinker. Size of latex particles are controlled by the surfactant.

Borbely, U.S. Appl. No.: 0030225190, among other things synthesis of styrene-ethylene glycol dimethacrylate (ST-EGDMA) was disclosed in solution. The polymer chains can grow without hindrance.

Size of nanoparticles was determined by Transmission Electron Microscopy (TEM) and Dynamic Laser Light Scattering (DLS). Reactivity of nanoparticles was measured by Nuclear Magnetic Resonance (¹H NMR).

According to measurements, it was found that reactivity of nanoparticles depended on the reaction time and the composition of the monomer feed.

Making of the nanocomposite. The nanoparticles were swollen in mixtures of dental monomers. Prepared nanoparticles in solution were mixed into the resin composed of vinyl monomers forming the modified resin, thus the shrinkage of the modified and generic dental resin arise only from the shrinkage of the resin (Refs. 6, 23).

Swelling of prepared nanoparticles in solution is better in the resin than prepared nanoparticles in emulsion.

Prismatic specimens are solidified from the modified and generic dental resin by blue light photopolymerization. The flexural properties of specimens are analyzed by an Instron Instrument and the surfaces of fracture of prismatic specimens are studied by Scanning Electron Microscope (SEM).

Volumetric shrinkage was measured by pycnometer method in water. Viscosities of nanocomposites were measured by Brookfield CAP 2000+ (at low viscosity CAP4 cone and plate, speed rate: 500-900 and at high viscosity CAP6 max 400 rpm).

The present invention is also directed to improved dental compositions which have reduced shrinkage. The compositions of the present invention are nanocomposites made from monomers that were mixed with reactive polymeric nanoparticles. The nanocomposites are made by synthesizing styrene-ethylene glycol-dimethacrylate (ST-EGDM) reactive nanoparticles in a homogeneous solution by free radical non-linear copolymerization. Once the ST-EGDM reactive nanoparticles were formed, they are reacted with a resin composition composed of vinyl monomers of commercial dental materials to form a modified resin. The modified resin was hardened using blue light photo-polymerization.

### BRIEF DESCRIPTION OF THE DRAWINGS

FIG. 1 is a representation of the reaction scheme for the preparation of organofiller of the modified dental nanocomposite of the present invention.

FIG. 2 is ¹H NMR spectra of reactive polymeric nanoparticles of the present invention.

FIGS. 3A and 3B are Dynamic Laser Light Scattering (DLS) and ¹H NMR spectra of reactive polymeric nanoparticles of the present invention respectively.

FIG. 4 shows the flexural properties of nanocomposite.

FIG. 5A-C, shows Flexural stress vs. flexure strain values for basic dental resin, Type-1 AD 15% (A60) and Type-2 BD 1,5%

Table 1 Conditions of preparation of RPNPs.

Table 2 Mean values of shrinkage and viscosity data of dental resin and modified resin with RPNPs.

### DETAILED DESCRIPTION OF THE INVENTION

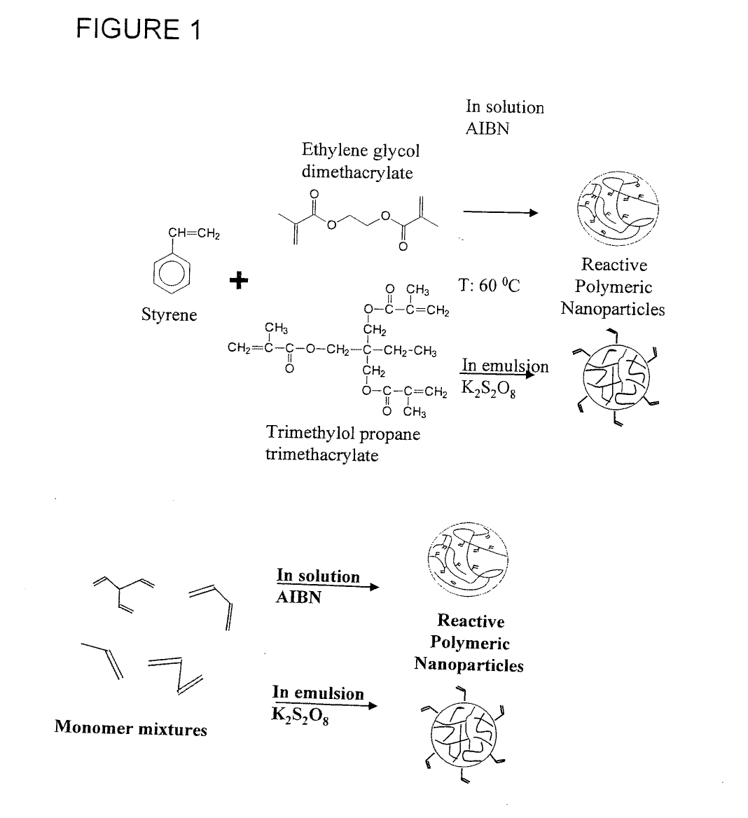

The general, steps of forming the nanoparticles of the present invention are shown in FIG. 1. Combination of mono/di/tri and multifunctional monomer (M₁) reactive polymeric nanoparticles (RPNPs) are formed in solution and miniemulsion by free radical non-linear copolymerization. The RPNPs as organofillers into the dental resin is added to mixtures of monomers and photoinitiator thus formed nanocomposites are investigated. Modified resins are filled nanoparticles which are formed from styrene and di and/or trifunctional acrylic monomers. Examples of the monovinyl acrylic monomers that can be used in the present invention include aromatic monovinyl monomers such as styrene methyl, styrene, ethyl styrene, chlorostyrene, vinyl benzoyl chloride, etc. and alphatic monovinyl monomers such as acrylic acid, methacrylic acid, ethyl acrylate, butyl acrylate, methyl methacrylate, ethyl methacrylate, butyl methacrylate, etc. Generic dental resin is filled nanoparticles which are formed from mono/di/tri or multifunctional vinyl acrylate dental monomers. Examples of the dental acrylic monomers that can be used 2,2′-bis[4-(2-hydroxy-3-methacryloyloxypropyl)phenyl]propane (Bis-GMA); ethoxylated Bisphenol A dimethacrylate (EBPDMA); 1,6-bis-[2-methacryloyloxyethoxycarbonylamino]-2,4,4-trimethylhexane (UDMA); dodecanediol dimethacrylate, stmethylol propane trimethacrylate (TMPTMA), 1,6-hexanediol dimethacrylate (HDDMA), 1,3-butanediol dimethacrylate (BDDMA), ethylene glycol dimethacrylate (EGDMA), diethylene glycol dimethacrylate (DEGDMA), triethylene glycol dimethacrylate (TEGDMA) or tetraethylene glycol dimethacrylate.

The pendant double bonds of these monomers form primary and secondary loops followed by branching and crosslinking. The final nanoparticles contain double bonds which are susceptible to further thermal or photo-activated post polymerization.

In accordance with the present invention, the dental resin was modified by reactive polymeric nanoparticles. The nanoparticles are formed by miniemulsion and/or homogenous copolymerization of a mono vinyl styrenic monomer such as styrene (ST) and a di- and trivinyl acrylic monomer. One suitable divinyl monomer is ethylene glycol dimethacrylate (EGDMA) and trivinyl monomer is trimethylol propane trimethacrylate (TMPTMA). The reaction may also include one or more surfactants and/or initiators. The composition (monomer ratio and concentration in feed) and initiator concentration are optimized to achieve high yield without gelation. The emulsifier concentration is also optimized. The preferred surfactant is sodium dodecyl sulphate. The preferred initiator is azoisobutironitrile (AIBN). Circumstances of preparation are shown by Table 1 (Table 1).

Preparation of ST-EGDMA (Type-1) copolymer, as, model additives to the dental resin is as follows: to the solution (toluene) azoisobutironitrile (AIBN) initiator, styrene and ethylene glycol dimethacrylate was added at 60° C.

Preparation of ST-TMPTMA (Type-2) copolymer as model additives to the dental resin is as follows: to the distilled water sodium dodecyl sulphate as surfactant and potassium peroxide as initiator, styrene and trimethylol propane trimethacrylate are added. The temperature of the free radical non-linear copolymerization is 60° C.

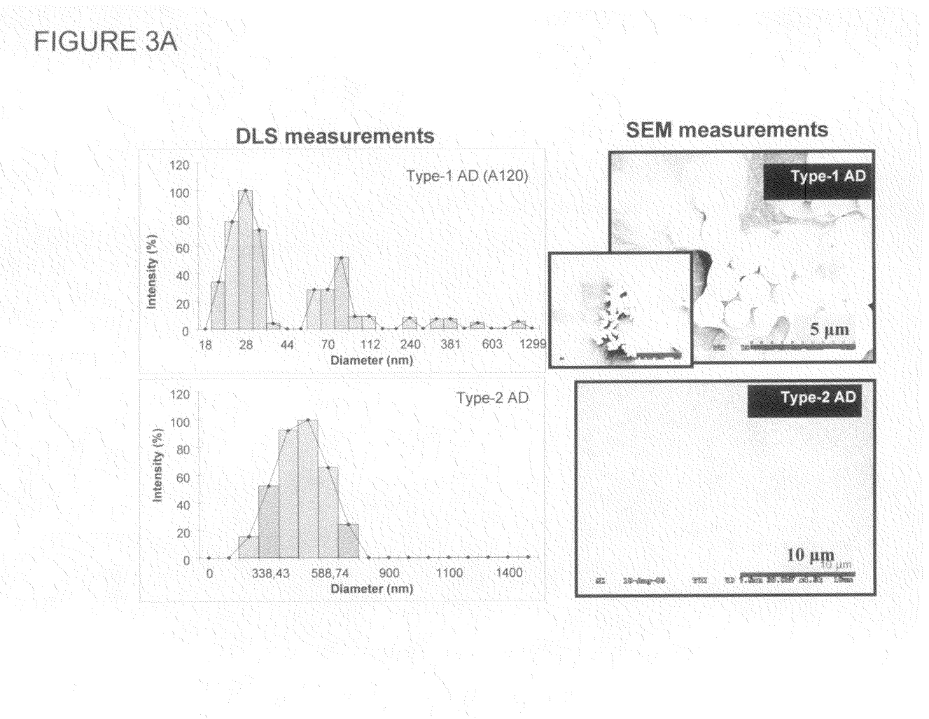

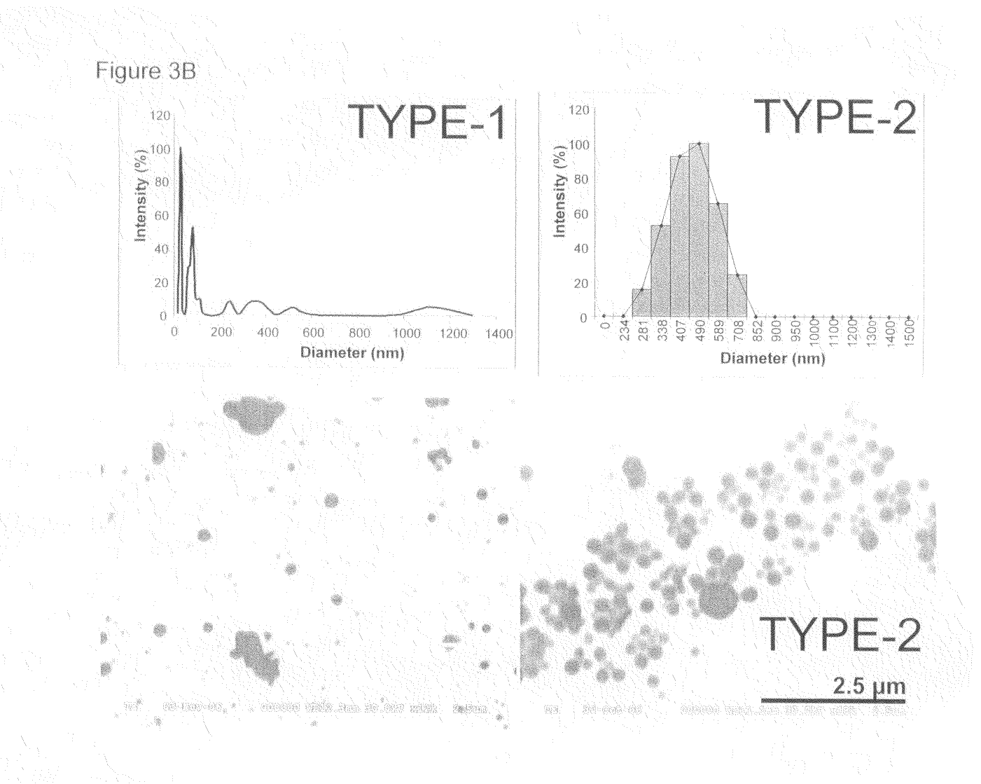

The prepared nanoparticles from solution (Type-1) and latex (Type-2) can be precipitated by adding, for example, an excess of methyl alcohol. The copolymer can be purified by dissolving three times in toluene and re-precipitate with methyl alcohol. Size, reactivity and structure of nanoparticles are analyzed by Dynamic Laser Light Scattering (DLS) and Nuclear Magnetic Resonance Spectroscopy (¹H NMR). Particle size was determined by Scanning Electron Microscopy (SEM). SEM measurements were performed on a Hitachi 3000N instrument to determine the particle size of RPNPs. Selected SEM micrographs are shown in FIG. 3, demonstrating the individual spherical particles (FIG. 3).

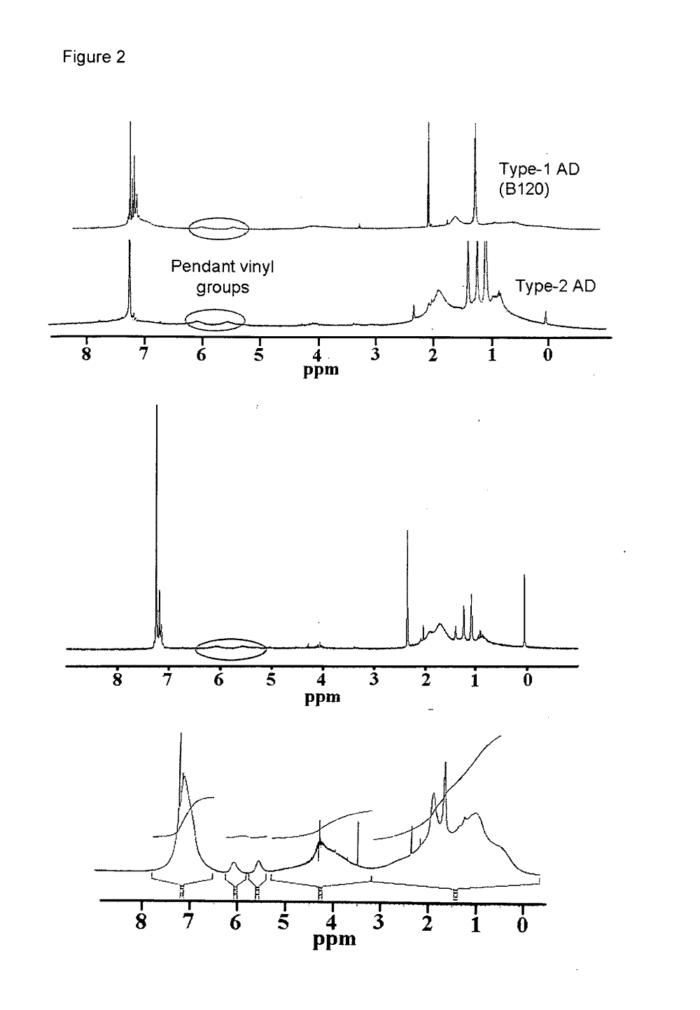

¹H NMR experiments were performed on Bruker 200 WP instrument at 200 MHz operating frequency in CDCl₃ solution at an RPNP concentration of 20 mg/ml. Peaks assigned for the aromatic region of the M₁ monomer were in the range of 6.3-7.3 ppm, and aliphatic protons at 0.6-2.3 ppm. Signals of reactive pendant vinyl groups were in the range of 5.5-6.2 ppm (FIG. 2).

Hydrodynamic diameter (HD) of nanoparticles was determined by DLS measurements using BI-200SM Brookhaven photometer equipped with a NdYAg solid state laser at an operating wavelength of λₒ=532 nm.

The size of the particles depended upon two parameters: (i) increasing the crosslinker (EGDMA, TMPTMA) content resulted in an increased particle size, (ii) the crosslinking ratio of larger particles is higher resulting in lower swelling ratio.

The nanoparticles can display swellable features in organic solvent, a very important factor in order to study dental resins consisting of mixtures of monomers.

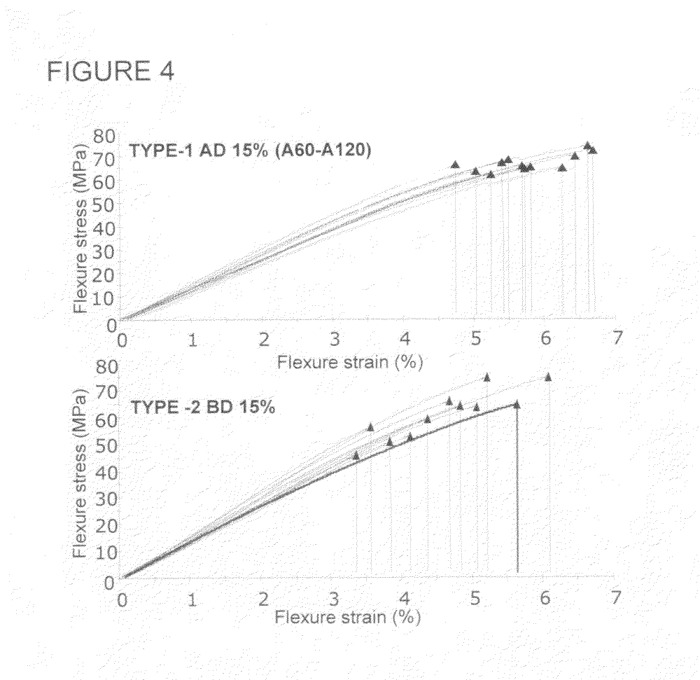

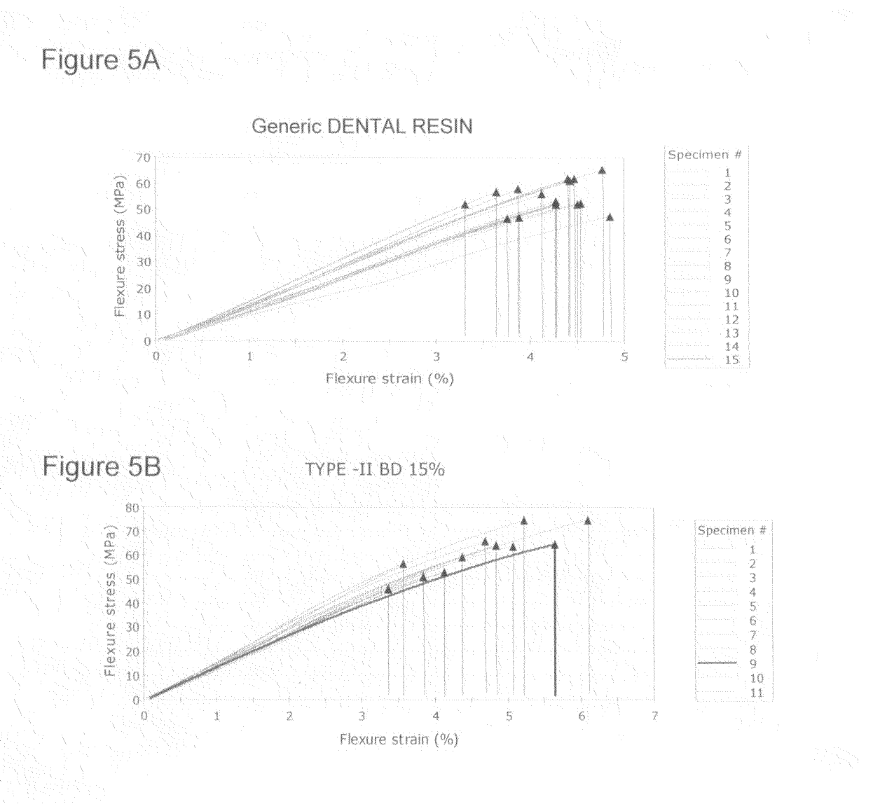

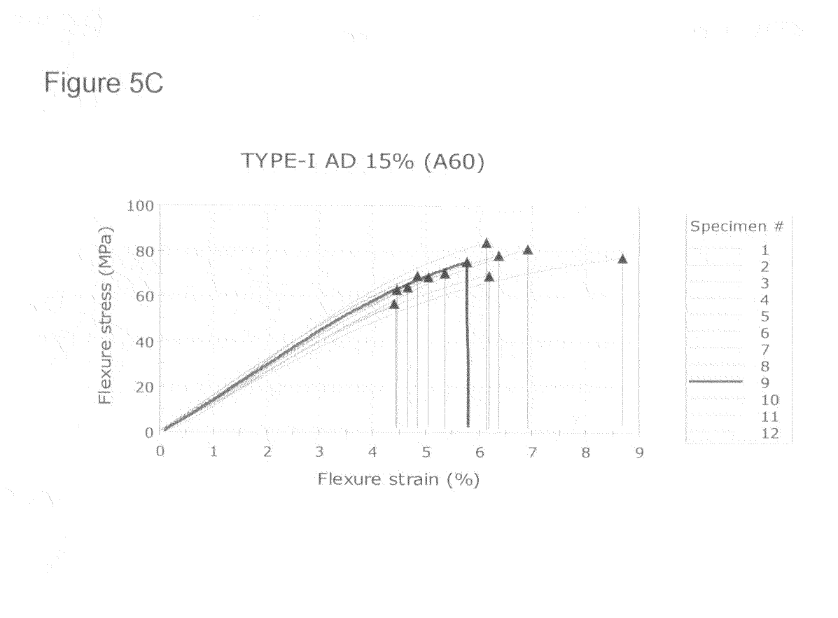

The Type-1 nanoparticles are dissolved in toluene which causes significant swelling. Toluene is added to the nanoparticles and the dental resin. After mixing, the solution is removed in vacuo until the weight is constant. The flexural properties (FIG. 4), viscosities and volumetric shrinkage of nanocomposites are analyzed (Table 2).

In accordance with the present invention nanoparticles as organofillers are prepared by using mono/di/tri and multifunctional monomer, surfactant at the preparation in emulsion and more types of initiator.

### Monovinyl Monomers:

Specific examples of suitable co-monomers that are useful to form a linear vinyl monomer include the following: (meth)acrylate esters such as methyl (meth)acrylate, ethyl (meth)acrylate, n-propyl (meth)acrylate, n-butyl (meth)acrylate, iso-propyl (meth)acrylate, iso-butyl (meth)acrylate, tertiary butyl (meth)acrylate, neopentyl-(meth)acrylate, iso-penthyl (meth)acrylate, n-amyl (meth)acrylate, n-heptyl (meth)acrylate, iso-heptyl (meth)acrylate, n-hexyl (meth)acrylate, n-octyl (methacrylate), iso-octyl (methacrylate), iso-amyl (meth)acrylate, 2-hydroxyethyl (meth)acrylate, 2-hydroxypropyl (meth)acrylate, N,N-dimethylaminoethyl (meth)acrylate, N,N-diethylaminoethyl (meth)acrylate, t-butylaminoethyl (meth)acrylate, 2-sulfoethyl (meth)acrylate, trifluoroethyl (meth)acrylate, glycidyl (meth)acrylate, benzyl (meth)acrylate, allyl (meth)acrylate, 2-n-butoxyethyl (meth)acrylate, 2-chloroethyl(meth)acrylate, sec-butyl-(meth)acrylate, tert-butyl (meth)acrylate, 2-ethylbutyl(meth)acrylate, cinnamyl(meth)acrylate, crotyl (meth)acrylate, cyclohexyl (meth)acrylate, cyclopentyl (meth)acrylate, 2-ethoxyethyl (meth)acrylate, furfuryl (meth)acrylate, hexafluoroisopropyl (meth)acrylate, methallyl (meth)acrylate, 3-methoxybutyl(meth)acrylate, 2-methoxybutyl (meth)acrylate, 2-nitro-2-methylpropyl (meth)acrylate, 2-ethylhexyl (meth)acrylate, lauryl (meth)acrylate, 2-phenoxyethyl (meth)acrylate, 2-phenylethyl (meth)acrylate, phenyl(meth)acrylate, propargyl (meth)acrylate, tetrahydrofurfuryl (meth)acrylate, norbornyl(meth)acrylate, tetrahydropyranyl (meth)acrylate, vinyl acetate, (meth)acrylonitrile, vinylpropionate, vinylidene chloride, (meth)acrylamide, N-methylolacrylamide, benzyl (meth)acrylate, iso-octyl (methacrylate), 2-ethylhexyl (meth)acrylate, octadecyl methacrylate, octadecyl acrylate, nonyl (meth)acrylate, iso-nonyl (meth)acrylate, decyl (meth)acrylate, iso-decyl (meth)acrylate, undecyl (meth)acrylate, iso-undecyl (meth)acrylate, tridecyl (meth)acrylate, iso-tridecyl (meth)acrylate, tetradecyl (meth)acrylate, iso-tetradecyl (meth)acrylate, lauryl (meth)acrylate, iso-lauryl (meth)acrylate, hydroxyethyl (meth)acrylate, hydroxyhexyl (meth)acrylate, hydroxyoctadecyl (meth)acrylate, hydroxylauryl (meth)acrylate, phenethyl (meth)acrylate, 6-phenylhexyl (meth)acrylate, phenyllauryl (meth)acrylate, 3-nitrophenyl-6-hexyl methacrylate, 3-nitrophenyl-18-octadecyl acrylate, ethyleneglycol dicycopentyl ether acrylate, vinyl ethyl ketone, vinyl propyl ketone, vinyl hexyl ketone, vinyl octyl ketone, vinyl butylketone, cyclohexyl acrylate, 3-methacryloxypropyldimethylmethoxysilane, 3-methacryloxypropylmethyldimethoxysilane, 3-methacryloxypropylpentamethyl-disiloxane, 3-methacryloxypropyltris(trimethylsiloxy)silane, 3-acryloxypropyldimethylinethoxysilane, acryloxypropylmethyldimethoxysilan, trifluoromethyl styrene, trifluoromethyl (meth)acrylate, tetrafluoropropyl (meth)acrylate, heptafluorobutyl methacrylate, N,N-dihexyl acrylamide, N,N-dioctyl acrylamide, aminoethylacrylate, aminoethyl methacrylate, aminoethyl butacrylate, aminoethylphenyl acrylate, aminopropyl (meth)acrylate, aminoisopropyl (meth)acrylate, aminobutyl (meth)acrylate, aminohexyl (meth)acrylate, aminooctadecyl (meth)acrylate, aminolauryl (meth)acrylate, N,N-dimethyl-aminoethyl (meth)acrylate, N,N-diethylaminoethyl (meth)acrylate, piperidino-N-ethyl acrylate, vinyl propionate, vinylacetate, vinyl butyrate, vinyl butyl ether, vinyl propyl ether, styrene and alkyl derivatives.

### Examples of Some Suitable Di- and Multivinyl Monomers:

Crosslinking monomers suitable for use as the cross-linker in the core polymer are known to those skilled in the art, and are generally di- and higher multifunctional monomers copolymerizable with the other core monomers, as for example, glycol dimethacrylates and acrylates, triol triacrylates and methacrylates and the like. The preferred crosslinking monomers are butylene glycol diacrylates.

### Some Specific Examples of Crosslinking Monomers are:

N,N′-methylene-bis-acrylamide, ethylene glycol di(meth)acrylate (EGD(M)A), diethylene glycol di(meth)acrylate, triethylene glycol di(meth)acrylate, tetraethylene glycol di(meth)acrylate, polyethylene glycol di(meth)acrylate, propylene glycol di(meth)acrylate, hexanediacrylate, cyclohexanedimethanoldivinil ester, polypropylene glycol di(meth)acrylate, butanediol di(meth)acrylate(BDDMA), hexanediol di(meth)acrylate, pentaerythritol tri(meth)acrylate, trimethylolethane tri(meth)acrylate, trimethylolpropane di(meth)acrylate, trimethylolpropane tri(meth)acrylate (TMPT(M)A), tripropylene glycol di(meth)acrylate; neopentyl glycol di(meth)acrylate, dipropylene glycol di(meth)acrylate, hacrylate, divinyl benzene, allyl (meth)acrylate (AL(M)A), divinyl benzene (DVB), glycidyl methacrylate, 2,2-dimethylpropane 1,3 diacrylate, 1,3-butylene glycol diacrylate, 1,3-butylene glycol dimethacrylate, 1,3-butadienol di(meth)acrylate, 1,4-butanediol di(meth)acrylate, 1,6-hexanediol di(meth)acrylate, polyethylene glycol 200 diacrylate, ethoxylated bisphenol A di(meth)acrylate, polyethylene glycol 600 dimethacrylate, poly(butanediol) diacrylate, trimethylolpropane tri(meth)acrylate, pentaerythritol tri(meth)acrylate (PETA), trimethylolpropane triethoxy tri(meth)acrylate, glyceryl propoxy tri(meth)acrylate, pentaerythritol tetra(meth)acrylate, and dipentaerythritol monohydroxypentaacrylate.

Special Monomers which are Used in Dental Resins:

Bis-phenol-A-dimethacrylate (Bis-EMA), bis-phenol-A-bis-glycidyl methacrylate (Bis-GMA), diethylene glycol dimethacrylate, triethylene glycol dimethacrylate (TEGMA), urethane dimethacrylate (UDMA), polyurethanedimethacrylate, diurethane dimethacrylate (DUDMA), polycarbonate dimethacrylate (PCDMA), ethoxylated bis-phenol-A-dimethacrylate (EBPDMA) diethylaminoethyl (meth)acrylate, (commonly rderred to as “DEA-EMA”), dimethylamino ethyl methacrylate, diethylaminoethyl methacrylate (DEAEMA), ethyleneglycol dimethacrylate, tetramethylene glycol, dimethacrylate, trimethylol propyl trimethacrylate, 1,6-hexanediol dimethacrylate, 1,3-butanediol dimethacrylate, and the like.

Surfactants which are Used at Emulsion:
Anionic surfactants: salts of carboxylic acid, sulfates, salts of sulfuric acid, phosphates.
Cationic surfactants: ammonium salt, N-alkyl-pyridine salt.
Non-ionic surfactants: fatty acid ester of polyols.
Amphoteric surfactants: derivative of betains.

### Thermal Initiators:

αα′ Azoisobutironitrile(AIBN), 4,4′-Azobis(4-cyanovaleric acid), 1,1′-Azobis(cyclohexanecarbonitrile), 2,2′-Azobis(2-methylpropionitrile), Benzoyl peroxide reagent grade, 2,2-Bis(tert-butylperoxy)butane,
2,5-Bis(tert-butylperoxy)-2,5-dimethylhexane, Bis[1-(tert-butylperoxy)-1-methylethyl]benzene tert-Butyl hydroperoxide, tert-Butyl peracetate, tert-Butyl peroxide 98% Cumene hydroperoxide, Dicumyl peroxide, Lauroyl peroxide, Peracetic acid and Potassium persulfate.

### Photoinitiators:

Diphenyl(2,4,6-trimethylbenzoyl)phosphine oxide, 4,4′-Dimethoxybenzoin, Anthraquinone, Anthraquinone-2-sulfonic acid Sodium salt, Benzene-chromium(0) tricarbonyl, 4-(Boc-aminomethyl)phenyl isothiocyanate, Benzil, Benzoin purified, Benzoin ethyl ether, Benzoin isobutyl ether, Benzophenone, Benzoic acid meets, Benzophenone/1-hydroxycyclohexyl phenyl ketone, Benzophenone-3,3′,4,4′-tetracarboxylic dianhydride, 4-Benzoylbiphenyl, 2-Benzyl-2-(dimethylamino)-4′-morpholinobutyrophenone, 4,4′-Bis(diethylamino)benzophenone, Camphorquinone, 2-Chlorothioxanthen-9-one, 5-Dibenzosuberenone, 2,2-Diethoxyacetophenone, 4,4′-Dihydroxybenzophenone, 2,2-Dimethoxy-2-phenylacetophenone, 4-(Dimethylamino)benzophenone, 4,4′-Dimethylbenzil, 3,4-Dimethylbenzophenone, Diphenyl(2,4,6-trimethylbenzoyl)phosphine oxide/2-hydroxy-2-methylpropiophenone, 4′-Ethoxyacetophenone, 2-Ethylanthraquinone, Ferrocene, 3-Hydroxyacetophenone, 4′-Hydroxyacetophenone, 3-Hydroxybenzophenone, 4-Hydroxybenzophenone, 1-Hydroxycyclohexyl phenyl ketone, 2-Hydroxy-2-methylpropiophenone, 2-Hydroxy-2-methylpropiophenone, 2-Methylbenzophenone, 3-Methylbenzophenone, Methylbenzoylformate, 2-Methyl-4′-(methylthio)-2-morpholinopropiophenone, 9,10-Phenanthrenequinone, 4′-Phenoxyacetophenone, Thioxanthen-9-one, Triarylsulfonium hexafluorophosphate salts, 3-Mercapto-1-propanol, 11-Mercapto-1-undecanol, 1-Mercapto-2-propanol and 3-Mercapto-2-butanol.

### EXAMPLES

### Preparation of Nanoparticles as Organofillers

### Example 1

### Preparation of ST-EGDMA and ST-TMPTMA Reactive Polymeric Nanoparticles

Reactive polymeric nanoparticles (ST-EGDMA; Type-1) and (ST-TMPTMA; Type-2) was prepared by free radical non-linear precipitated copolymerization in emulsion (at Type-2) and homogenous solution (at Type-1). The monomer ratio in feed was from 1/9 to 9/1. The hydrophobic copolymers were formed as follows: 100-ml, tree necked, round bottom flask was equipped with paddle stirrer, thermometer, nitrogen inlet and reflux condenser under a nitrogen atmosphere. At preparation of Type-2 the emulsifier was sodium dodecyl sulphate, initiator was potassium peroxide. Azoisobutironitrile (AIBN) as initiator was used in preparation of nanoparticles in homogenous solution (toluene).

In emulsion: amount of monomers were 2.50 gram, which was added to the continuous phase (distilled water) consisting of emulsifier 0.6 grams, initiator 0.1 mol %. The emulsion was stabilized by ultasonication for 10 min.

In homogenous solution: the monomer concentration was 0.556 and 0.278 mol/dm³. The initiator ranges from 1 mol % to 10 mol %. The reaction time was different to achieve high yield. The temperature of polymerization is 60° C. At this value of temperature reactive initiator roots were generated from the initiator thus the chain propagation and forming of macromolecules were started. After the polymerization from the latex (at Type-2) and solution (at Type-1) the polymer was precipitated by three excess methyl alcohol and it was centrifuged and dryed. After this it was cleaned from the unreacted monomers by this way: the copolymer was dissolved or swollen in toluene and it was precipitated by methyl alcohol repeated three times.

The polymer structure and reactivity was characterized by NMR. Swelling behavior of nanoparticles and size and distributions was studied by Dynamic Laser Light Scattering (DLS) in solution and in dried form by Scanning Electron Microscopy (SEM). It was found that size of nanoparticles was influenced by the monomer composition and ratio in feed and reaction time.

### Example 2

### Preparation of MMA/EGDMA and MMA/TMPTMA Reactive Polymeric Nanoparticles

The copolymers were prepared alike to the Example 1.

### Example 3

### Preparation of MMA/EGDMA/HDDA and MMA/TMPTMA/HDDA Reactive Polymeric Nanoparticles

The copolymers were prepared alike to the Example 1.

### Example 4

### Preparation of MMA/Bis-GMA/EGDMA and MMA/Bis-GMA/TMPTMA Reactive Polymeric Nanoparticles

The copolymers were prepared alike to the Example 1.

### Example 5

### Preparation of MMA/TEGDMA Reactive Polymeric Nanoparticles

The copolymers were prepared alike to the Example 1.

### Example 6

### Preparation of EGDMA/TEGDMA Reactive Polymeric Nanoparticles

The copolymers were prepared alike to the Example 1.

### Example 7

### Preparation of TMPTMA/TEGDMA Reactive Polymeric Nanoparticles

The copolymers were prepared alike to the Example 1.

### Example 8

### Preparation of MMA/EGDMA/TEGDMA Reactive Polymeric Nanoparticles

The copolymers were prepared alike to the Example 1.

### Example 9

### Preparation of MMA/TMPTMA/TEGDMA Reactive Polymeric Nanoparticles

The copolymers were prepared alike to the Example 1.

### Example 10

### Preparation of MMA/EGDMA/TEGDMA/HDDA Reactive Polymeric Nanoparticles

The copolymers were prepared alike to the Example 1.

### Example 11

### Preparation of MMA/TMPTMA/TEGDMA/HDDA Reactive Polymeric Nanoparticles

The copolymers were prepared alike to the Example 1.

### Preparation of Nanocomposite (RESIN+RPNPs)

Prepared nanoparticles described examples 1-11 are mixed with dental resin. The dental resin consists of dental acrylic monomers and photoinitiator. Because of different swelling features of nanoparticles, which were influenced by the method of the preparation and the applied monomer mixtures, the forming of the nanocomposite is diverse. The prepared nanoparticles in miniemulsion the permeability of crosslinking network is not so good for the monomers of the dental resin. However the prepared nanoparticles in homogenous solution this feature is better because of the polymer chain can grow without hindrance and after the precipitation it is remained. To achieved the homogenous distributions in the dental resin toluene and/or other organic solvent is necessary to add to the dental resin and the nanoparticles thus they are “pre-swelled” in solvent after this in dental resin.

Shrinkage, viscosity and flexural properties of prepared nanocomposites were studied. The optimal shrinkage and viscosity can be reached by the modification of nanoparticles (quantitative and qualitative). The optimal mechanical properties were ensured from the inorganic filler however it was expected that the flexural properties of the nanocomposite are better than dental resin without organic filler.

TABLE 1
Conditions of preparation of RPNPs in solution and in emulsion

| | | Ratio of | Monomer | | |
|---|---|---|---|---|---|
| | | monomers | concentra- | Initiator/ | Reaction |
| No of | | (M₁/M₂) | tion | monomers | time |
| sample | Sample | (mol %) | (mol/dm³) | (mol %) | (min) |
| Sample1 | Type-1 AD | 50:50 | 0.556 | 10 | 120 |
| | (B120) | | | | |
| Sample2 | Type-1 AD | 50:50 | 0.556 | 5 | 60 |
| | (A60) | | | | |
| Sample3 | Type-1 AD | 50:50 | 0.556 | 5 | 120 |
| | (A120) | | | | |
| Sample4 | Type-2 AD | 50:50 | 0.226 | 1 | 120 |
| Sample5 | Type-2 BD | 70:30 | 0.286 | 1 | 480 |

TABLE 2
Mean values of shrinkage and viscosity data of
dental resin and modified resin with RPNPs.

Mean values

| | | Polymerization | |
|---|---|---|---|
| Number of | | Shrinkage | Viscosity |
| sample | Sample | (%) | (Pas) |
| Sample 1 | Generic | 6.3 | 0.532 |
| | Dental Resin | | |
| Sample 2 | NCDR with | 6.1 | 23.996 |
| | Type-1 AD | | |
| | (B120)(15%) | | |
| Sample 3 | NCDR with | 5.4 | 9.208 |
| | Type-1 AD | | |
| | (A60)(15%) | | |
| Sample 4 | NCDR | 4.5 | 14.194 |
| | Type-1 AD | | |
| | (A60-A120) | | |
| | (15%) | | |
| Sample 5 | NCDR with | 7.1 | 0.892 |
| | Type-2 AD | | |
| | (10%) | | |
| Sample 6 | NCDR with | 6.9 | 0.961 |
| | Type-2 BD | | |
| | (10%) | | |
| Sample 7 | NCDR | 5.8 | 1.565 |
| | Type-2 BD | | |
| | (15%) | | |

What is claimed:

1. An improved dental filling material comprising the reaction product of an acrylic dental resin with a nanocomposite, said nanocomposite comprising the reaction product of a first monomer comprised of a mono/di/tri and/or multi-vinyl acrylic compound and a second monomer comprised of an acrylic or styrenic based compound.

2. The dental filling material according to claim 1 wherein said nanocomposite is formed by free radical non-linear copolymerization.

3. The dental filling material according to claim 2 wherein said reaction is in an emulsion.

4. The dental filling material according to claim 3 wherein said reaction is in a homogeneous solution.

5. The dental filling material according to claim 2 wherein said second monomer is styrene.

6. The dental filling material according to claim 2 wherein said second monomer is an acrylic compound.

7. The dental filling material according to claim 2 wherein said first monomer is EGDMA.

8. The dental filling material according to claim 2 wherein said second monomer is TMPTMA.

9. A nanocomposite comprising the reaction product of reactive polymeric nanoparticles and an acrylic dental resin.

10. The nanocomposite according to claim 9 wherein the said nanoparticles are formed from a mono/di/tri or multivinyl monomer.

11. The nanocomposite according to claim 10 wherein the said monovinyl monomer is styrenic.

12. The nanocomposite according to claim 10 wherein the said di and trivinyl monomer is acrylic.

13. The nanocomposite according to claim 10 wherein the said divinyl monomer is ethylene glycol dimethacrylate (EGDMA).

14. The nanocomposite according to claim 10 wherein the said trivinyl monomer is trimethylol propane trimethacrylate (TMPTMA).

15. A nanocomposite comprising the reaction product of a first monomer comprised of a mono/di/tri and/or multi-vinyl acrylic compound and a second monomer comprised of an acrylic or styrenic based compound.

16. The nanocomposite according to claim 15 wherein said nanocomposite is formed by free radical non-linear copolymerization.

17. The nanocomposite according to claim 16 wherein said reaction is in an emulsion.

18. The nanocomposite according to claim 16 wherein said reaction is in a homogeneous solution.

19. The nanocomposite according to claim 16 wherein said second monomer is styrene.

20. The nanocomposite according to claim 16 wherein said second monomer is an acrylic compound.

21. The nanocomposite according to claim 16 wherein said first monomer is EGDMA.

22. The nanocomposite according to claim 16 wherein said second monomer is TMPTMA.

23. The nanocomposite of claim 16 reacted with a dental acrylic material.

24. The polymeric nanoparticles according 18 wherein the solvent is toluene.

25. The polymeric nanoparticle according to claim 16 wherein said nanoparticle is formed by reaction product of mono/di/tri and multivinyl styrenic and/or acrylic monomers.

26. The polymeric nanoparticle according to claim 1 wherein said weight percent of the nanoparticles to the dental resin ranges from about 10% to about 90% by weight.

27. The polymeric nanoparticle according to claim 11, wherein the reaction further comprises one or more initiators.

28. The polymeric nanoparticle according to claim wherein the reaction further comprises one or more surfactants.

16. The polymeric nanoparticle according to claim 15 wherein said surfactant is sodium dodecyl sulfate.

17. The polymeric nanoparticle according to claim 11a wherein said initiator is potassium peroxide.

18. The polymeric nanoparticle according to claim 11c wherein said initiator is azoisobutironitrile (AIBN).

19. A method of forming reactive polymeric nanoparticles comprising reacting a mono vinyl styrenic monomer with a di/trivinyl acrylic monomer in miniemulsion and homogenous solution.

20. The method according to claim 19 wherein said reaction is a free radical non-linear copolymerization.

21. The method according to claim 19 wherein said mono vinyl styrenic monomer is a styrene.

22. The method according to claim 19, wherein said divinyl acrylic monomer is a ethylene glycol dimethacrylate (EGDMA).

23. The method according to claim 19, wherein said trivinyl acrylic monomer is a trimethylol propane trimethacrylate (TMPTMA).

24. The method according to claim 19, wherein one or more surfactants is added to the reaction.

25. The method according to claim 19, wherein one or more initiators are added to the reaction.

26. The method according to claim 25, wherein said surfactant is sodium dodecyl sulfate.

27. The method according to claim 25, wherein said initiator is potassium peroxide.

28. The method according to claim 25, wherein said initiator is a azoisobutironitrile (AIBN).

29. The method according to claim 19 wherein the formed nanoparticles has swellable properties.
